(19) United States
(12) Patent Application Publication
(10) Pub. No.: US 2012/0184824 A1
(43) Pub. Date: Jul. 19, 2012

(54) METHOD FOR MEASURING THE LOCAL STIFFNESS INDEX OF THE WALL OF A CONDUCTING ARTERY, AND CORRESPONDING EQUIPMENT

(76) Inventors: Mathieu Collette, Beaufort en Vallee (FR); Anne Humeau, Angers (FR); Georges Leftheriotis, Rochefort Sur Loire (FR)

(21) Appl. No.: 13/140,730

(22) PCT Filed: Dec. 18, 2009
(86) PCT No.: PCT/EP2009/067593

§ 371 Date: Mar. 29, 2012

(30) Foreign Application Priority Data
Dec. 19, 2008 (FR) 0807264

(51) Int. Cl. A61B 5/053; A61B 5/00

(52) U.S. Cl. 600/301; 600/547

(57) ABSTRACT

Disclosed herein is a method for measuring the local stiffness index of the wall of a conducting artery carrying the blood of a patient. The method includes a step of measuring, at a single measurement point, the electric impedance variation of a volume of the blood flowing in a segment of the artery; a step of determining a first intermediate index representative of a resistive characteristic involved in the stiffening of the wall, and a second intermediate index representative of a capacitive characteristic involved in the stiffening of the wall, the first and second intermediate indices being obtained from the measure of the electric impedance variation; and a step of determining the local stiffness index based on the first and second intermediate indices.

### CROSS-REFERENCE TO RELATED APPLICATIONS

This application is a national stage application under 35 U.S.C. 371 of International Patent Application Serial No. PCT/EP2009/067593, filed Dec. 18, 2009, entitled “METHOD FOR MEASURING THE LOCAL STIFFNESS INDEX OF THE WALL OF A CONDUCTING ARTERY AND CORRESPONDING EQUIPMENT,” which claims priority from French Patent application Ser. No. 08/07264, filed Dec. 19, 2008, the disclosures of each are herein incorporated by reference in their entirety.

### FIELD OF THE INVENTION

The field of the invention is that of the techniques for determining the stiffness of the conducting arteries of human beings or animals. More specifically, the invention relates to a method and equipment for determining the local stiffness index of the wall of a conducting artery carrying the blood of a patient.

### BACKGROUND OF THE INVENTION

Cardiovascular diseases currently remain the primary cause of death in developed countries. This is related, in particular, to the fact that a constant increase in the cardiovascular risk factors is being observed in the populations thereof.

A large number of studies have shown a strong association between the risk of a cardiovascular accident and alterations in the vascular parietal structures and/or functions. Stiffening of the vascular tree is physiologically linked with age and accelerates with the development of artheromatous disease promoted by risk factors in the ranks of which are included diabetes, hypertension, tobacco consumption, hypercholesterolemia, heredity, sedentariness . . . .

Arteriosclerosis remains asymptomatic for a long time over the first decades of life, being later revealed by a symptom or an acute and sometimes fatal event.

In order to reduce the harmful effects of degenerative diseases of the vascular system, and in particular arteriosclerosis, it is therefore necessary to improve the screening thereof, so as to prevent the appearance of same, or at least anticipate or stop the development thereof by early patient management.

Sclerosis of the artery wall is most often accompanied by an increase in the stiffness of the artery wall.

To date, various techniques can be implemented for the purpose of determining the stiffness of a conducting artery.

A regional stiffness index of the aortic artery wall can be obtained by measuring the conduction speed of the pulse wave (in metres per second) by tonometry at two points (carotid and femoral). This non-traumatic technique is currently considered to be the reference. However, the routine use of same still remains tedious and delicate, and the results obtained depend in large part on the expertise of the operator and the morphology of the patient. In addition, this technique only enables the aorta to be analysed, the main elastic artery of the body, and enables only a regional index and not a local index of the stiffness of the artery to be obtained.

A regional stiffness index is an index representative of the stiffness of an entire artery. In contrast, a local stiffness index is an index representative of the stiffness of a portion (or a segment) of an artery.

Ultrasound techniques (e.g., such as ultrasonography) likewise enable vascular compliance (elasticity) to be assessed. Proper implementation of these techniques depends, however, on the expertise of the operator, and remains entirely manual. Furthermore, although they offer useful morphological information (viewing of the artery and the walls thereof), they cannot be proposed as part of the routine screening and diagnosis of cardiovascular diseases, due to the cost thereof and the length of each examination.

Another technique consists in studying the morphology of the arterial pressure signal reflecting waves recorded on the finger, so as to determine the stiffness of the arteries. This technique only enables a regional stiffness index of the arterial tree to be obtained and not a local stiffness index of an artery.

Japanese patent application JP2003169779 describes another technique which consists in measuring the velocity of propagation of an impedance wave carried in an artery, so as to estimate the conduction speed of the pulse wave, and to deduce therefrom a local stiffness index of the aortic wall. This technique has the disadvantage, in particular, of requiring consecutive recording of the impedance signal at two separate anatomical sites, so as to determine the local stiffness index of an artery wall.

### SUMMARY OF THE INVENTION

For the most part, therefore, these techniques of the prior art have the following disadvantages:
they are relatively difficult to implement and require a certain level of expertise;
they are costly to implement.

In addition, it is known that stiffening of the wall of an artery can result from various characteristics.

Stiffening of the wall of an artery can, in particular, result from a so-called resistive characteristic which is the result of an increase in intramural pressure related to with an increase in peripheral resistance. Peripheral resistance is defined as the ratio between the differential pressure (i.e., the systolic pressure from which the diastolic pressure is subtracted) and the arterial flow rate. Peripheral resistance opposes the blood flow in the artery in the systolic phase, which results in an increase in the differential pressure inside the artery. The increase in pressure tends to cause the artery to dilate. In the case where an artery is dilated to the maximum, i.e., the radius thereof can no longer be increased, the artery appears to be stiff. The resistive component is representative of this effect.

Stiffening of the wall of an artery can also result from a decrease in the so-called capacitive characteristic thereof, which results from the capacity of an artery to store mechanical energy, due to the deformation of the artery during the systolic phase, and to restore same during the diastolic phase.

However, the techniques of the prior art lead only to the obtainment of an index representative of the regional or local stiffness of an artery, without providing any information about the characteristics involved in said stiffening or about the significance thereof.

The aim of the invention, in particular, is to overcome said disadvantages of the prior art.

More specifically, one aim of the invention is to provide a technique for determining the local stiffness of a conducting artery carrying the blood of a patient.

The invention aims, in particular, to provide such a technique which enables the influence of at least some characteristics involved in the stiffening of an artery to be known.

The invention likewise aims to provide such a technique which is reliable and accurate.

Another aim of the invention is to produce such a technique which is simple to implement.

Another aim of the invention is to carry out the measurement on a single anatomical site.

The invention also as the aim of providing such a technique which is relatively inexpensive to implement.

### BRIEF DESCRIPTION OF THE FIGURES

Other characteristics and advantages of the invention will become more apparent upon reading the following description of a preferred embodiment, which is given for purely illustrative and non-limiting purposes, and from the appending drawings, in which:

FIG. 1 is a schematic representation of equipment for implementing a method according to the invention, wherein the electrodes are positioned so as to determine the stiffness of the walls of the aorta;

FIG. 2 shows a positioning of the electrodes for determining the stiffness of the femoral artery;

FIG. 3a is a curve showing the electrocardiogram (ECG) of a patient;

FIG. 3b is a curve showing the inverse of the impedance variation in a volume (V) of blood flowing in a portion of an artery placed between the emitting and receiving electrodes of equipment according to the invention;

FIG. 3c is a curve showing the derivative of the curve shown in FIG. 3b; and

FIG. 4 shows a flowchart of a method according to the invention.

### DETAILED DESCRIPTION

These aims, as well as others which will become apparent hereinafter, are achieved by means of a method for measuring the local stiffness index (Ira) of the wall of a conducting artery carrying the blood of a patient.

According to the invention, such a method includes at least:
a step of measuring, at a single measurement point, the electric impedance variation (ΔZ) of a volume (V) of the blood flowing in a segment of said artery;
a step of determining a first intermediate index (RP %, RP) representative of a resistive characteristic involved in the stiffening of said wall, and a second intermediate index (PCPA %, ID) representative of a capacitive characteristic involved in the stiffening of said wall, the first (RP %, RP) and second (PCPA %, ID) intermediate indices being obtained from the measure of the electric impedance variation (ΔZ);
a step of determining said local stiffness index (Ira) based on said first (RP %, RP) and second (PCPA %, ID) intermediate indices.

The invention is thus based on a completely novel and inventive approach which consists in determining at least two intermediate indices each representative of a resistive characteristic and a capacitive characteristic involved in the stiffening of an artery, and in then determining a global index of the local stiffness of an artery wall based on the predetermined intermediate indices.

The inventors discovered that the stiffening of an artery wall can, in particular, result from a so-called resistive characteristic and a so-called capacitive characteristic. Defining the local stiffness of an artery wall therefore assumes an assessment of the resistive and capacitive characteristics involved in the overall stiffening of the wall of an artery. Knowing each of these resistive and capacitive characteristics enables a local stiffness index of an artery wall to be determined, which is particularly accurate and representative of reality.

The resistive characteristic expresses an increase in the ratio between the intramural pressure and the arterial flow rate. For example, this can be a matter of peripheral or local resistance.

The capacitive characteristic results in the capacity of an artery to store mechanical energy due to the deformation of the artery in the systolic phase and to restore it in the diastolic phase. It is therefore related to the elasticity of the artery. For example, this can be a matter of the distensibility of the artery.

Implementation of the invention thus enables:
a global index to be obtained, which enables the local stiffness of an artery wall to be known accurately and realistically, the recognition of which makes it possible to know if, at one point, the artery is rather stiff or rather flexible, and
two intermediate indices to be obtained, the recognition of which makes it possible to know the respective significance of a resistive characteristic and a capacitive characteristic of the artery involved in the local stiffness thereof.

Implementation of the invention therefore enables the person responsible for analysing the results obtained to have a more accurate picture of the stiffness of a segment of an artery, and in particular to know the significance of the various characteristics which are at the source thereof. This knowledge can subsequently enable a patient to be treated more effectively, e.g., by administering thereto a treatment which is targeted at each of the characteristics involved in the stiffening of the arteries of same.

Furthermore, implementation of the invention only requires measurement of a impedance variation in a volume of blood flowing in a segment of an artery, and does not require, as is the case according to the prior art, consecutively carrying out two measurements of the impedance variation at two separate anatomical sites. The present invention is therefore relatively simple to implement.

According to a first advantageous embodiment, said first intermediate index (RP %) is an index representative of the peripheral resistance downstream from said segment during a systolic phase of a heartbeat, and said second intermediate index (PCPA %) is an index representative of the capacity of said artery to store mechanical energy due to the deformation of said artery during said systolic phase of said heartbeat, and to restore same during the diastolic phase of said heartbeat.

Implementation of the invention therefore enables indices to be obtained which are representative of the so-called resistive and so-called capacitive characteristics involved in the stiffening of the artery, and about the significance thereof in said stiffening.

Said step of determining said local stiffness index (Ira) preferably includes a calculation step according to the formula:

Ira=(1−|PCPA %|)·RP %+(1−RP %)·|PCPA %|

This formula enables an index to be efficiently and accurately determined from the two intermediate indices, which is representative of the local stiffness of the wall of an artery segment.

A method according to the invention advantageously includes a step of calculating said intermediate index (PCPA %) according to the formula:

$${PCPA}_{\%} = \frac{J - I}{J + I} \cdot 100$$

$$with I = {\int }_{{t}_{1}}^{{t}_{2}} \vert \frac{d}{d t} ( \frac{1}{Δ Z} ) \vert d t and J = {\int }_{{t}_{2}}^{{t}_{3}} \vert \frac{d}{d t} ( \frac{1}{Δ Z} ) \vert d t ,$$

t₁ representing the appearance time of the base of the derivative of the impedance variation

$$( \frac{d}{d t} \frac{1}{Δ Z} )$$

from the start of said systolic phase,

t₂ representing the appearance time of the maximum of the derivative of the impedance variation

$$( \frac{d}{d t} \frac{1}{Δ Z} )$$

from the start of said systolic phase,

t₃ representing the appearance time of the intersection of the derivative of the impedance variation

$$( \frac{d}{d t} \frac{1}{Δ Z} )$$

and a straight line parallel to the x-axis passing through the point of the curve

$$( \frac{d}{d t} \frac{1}{Δ Z} )$$

at time t₁.

This formula enables an index to be efficiently and accurately determined, from the measurement of impedance variation, which is representative of the capacitive characteristic of the artery.

According to another advantageous characteristic, a method according to the invention includes a step of calculating said first intermediate index (RP %) according to the formula:

$${RP}_{\%} = \frac{K - I}{K} \cdot 100$$

K being a constant dependent on means implemented to carry out said step of measuring the electric impedance variation (ΔZ).

This formula enables an index to be efficiently and accurately determined from the measurement of impedance variation, which is representative of the resistive characteristic of the artery.

According to a second advantageous embodiment, said first intermediate index is an index (RP) which is representative of the local resistance of said segment during a systolic phase of a heartbeat, and said second intermediate index is an index (ID) which is representative of the distensibility of said artery during a systolic phase of a heartbeat.

Implementation of the invention therefore enables indices to be obtained which are representative of the so-called resistive and so-called capacitive characteristics involved in the stiffening of the artery, and about the significant thereof in said stiffening.

In this case, a method according to the invention preferably includes a step of measuring the arterial pressure in the systolic phase (PAS), the arterial pressure in the diastolic phase (PAD), and calculating the average arterial pressure (PAM). Said step of determining said local stiffening index (Ira) advantageously includes a step of calculating according to the formula:

$$Ira = \frac{PAS - PAD}{PAM} \cdot \frac{RP \cdot ID}{RP + ID}$$

This formula enables an index to be efficiently and accurately determined from the two intermediate indices, which is representative of the local stiffness of the wall of an artery segment.

A method according to the invention preferably includes a step of calculating said first intermediate index (RP) according to the formula:

$$RP = \frac{PAM}{( I + J )}$$

$$with I = {\int }_{{t}_{1}}^{{t}_{2}} \vert \frac{d}{d t} ( \frac{1}{Δ Z} ) \vert d t and J = {\int }_{{t}_{2}}^{{t}_{3}} \vert \frac{d}{d t} ( \frac{1}{Δ Z} ) \vert d t ,$$

t₁ representing the appearance time of the base of the derivative of the impedance variation

$$( \frac{d}{d t} \frac{1}{Δ Z} )$$

from the start of said systolic phase,

t₂ representing the appearance time of the maximum of the derivative of the impedance variation

$$( \frac{d}{d t} \frac{1}{Δ Z} )$$

from the start of said systolic phase,

t₃ representing the appearance time of the intersection of the derivative of the impedance variation

$$( \frac{d}{d t} \frac{1}{Δ Z} )$$

and a straight line parallel to the x-axis passing through the point of the curve

$$( \frac{d}{d t} \frac{1}{Δ Z} )$$

at time t₁.

This formula enables an index to be efficiently and accurately determined, from the measurement of impedance variation, which is representative of the resistive characteristic of the artery.

A method according to the invention preferably includes a step of calculating said second intermediate index (ID) according to the formula:

$$ID = PAM \frac{100 [ 2 ( J - I ) - ( J + I ) ]}{{( J + I )}^{2}}$$

This formula enables an index to be efficiently and accurately determined, from the measurement of the impedance variation, which is representative of the capacitive characteristic of the artery.

According to a particular embodiment, a method according to the invention includes a step of acquiring an electrocardiogram (ECG) signal from said patient, and a step of synchronising said electrocardiogram ECG signal and said impedance variation (ΔZ).

In this way, the indices (Ira), (RP %, RP) and (PCPA %, ID) can be calculated for each heartbeat.

A method according to the invention preferably includes a plurality of:
steps of determining said first (RP %, RP) and said second (PCPA %, ID) intermediate indices;
steps of determining said local stiffness index (Ira) based on said first (RP %, RP) and said second (PCPA %, ID) intermediate indices,

said determination steps being carried out during consecutive heartbeats (R), said method also including a step of calculating the average of each of said indices (Ira), (RP %), (RP), (PCPA %), (ID) during said heartbeats (R).

This particular embodiment enables the accuracy of the results obtained to be improved.

A method according to the invention advantageously includes a plurality of steps of measuring, at a single measurement point, the electric impedance variation (ΔZ) of a volume (V) of blood flowing in a segment of said artery, each of said measurements being carried out on different heartbeats (R), said method also including a step of determining the average impedance variation on said heartbeats and a step of determining said first (RP %, RP) and said second (PCPA %, ID) based on said average.

This embodiment likewise enables the accuracy of the results obtained to be improved.

According to a preferred characteristic, a method according to the invention includes a step of displaying said local stiffness index (Ira) of the wall of a conducting artery, and a step of displaying said first (RP %, RP) and said second (PCPA %, ID) intermediate indices.

The measurement results can thus be used directly by a medical practitioner so as to assist same in diagnosing the clinical state of a patient, e.g., with a view to administering a suitable treatment thereto.

The invention likewise relates to equipment for implementing the method for determining the local stiffness index (Ira) of the wall of a blood-carrying conducting artery of a patient.

According to the invention, such equipment includes:
means for measuring, at a single measurement point, the electric impedance variation (ΔZ) of a volume (V) of the blood flowing in a segment of said artery;
means for determining a first intermediate index (RP %, RP) representative of a resistive characteristic involved in the stiffening of said wall, and a second intermediate index (PCPA %, ID) representative of a capacitive characteristic involved in the stiffening of said wall;
means for determining said local stiffness index (Ira) based on said first (RP %, RP) and second (PCPA %, ID) intermediate indices.

According to a first advantageous embodiment, said means for determining said first intermediate index (RP %) include means for determining an index representative of the peripheral resistance downstream from said segment during a systolic phase of a heartbeat, and said means for determining said second intermediate index (PCPA %) include means for determining an index representative of the capacity of said artery to store mechanical energy due to the deformation of said artery during said systolic phase of said heartbeat and to restore same during the diastolic phase of said heartbeat.

In this case, equipment according to the invention preferably includes means for calculating said local stiffness index (Ira) according to the following formula:

Ira=(1−|PCPA %|)·RP %+(1−RP %)·|PCPA %|

According to another advantageous characteristic, said means for calculating said second intermediate index (PCPA %) include means for calculating according to the formula:

$${PCPA}_{\%} = \frac{J - I}{J + I} \cdot 100$$

$$with I = {\int }_{{t}_{1}}^{{t}_{2}} \vert \frac{d}{d t} ( \frac{1}{Δ Z} ) \vert d t and J = {\int }_{{t}_{2}}^{{t}_{3}} \vert \frac{d}{d t} ( \frac{1}{Δ Z} ) \vert d t ,$$

t₁ representing the appearance time of the base of the derivative of the impedance variation

$$( \frac{d}{d t} \frac{1}{Δ Z} )$$

from the start of said systolic phase,

t₂ representing the appearance time of the maximum of the derivative of the impedance variation

$$( \frac{d}{d t} \frac{1}{Δ Z} )$$

from the start of said systolic phase,

t₃ representing the appearance time of the intersection of the derivative of the impedance variation

$$( \frac{d}{d t} \frac{1}{Δ Z} )$$

and a straight line parallel to the x-axis passing through the point of the curve

$$( \frac{d}{d t} \frac{1}{Δ Z} )$$

at time t₁.

Said means for calculating said first intermediate index (RP %) preferably include means for calculating according to the formula:

$${RP}_{\%} = \frac{K - I}{K} \cdot 100$$

K being a constant dependent on means implemented to carry out said step of measuring the electric impedance variation (ΔZ).

According to a second advantageous embodiment, said means for determining said first intermediate index (RP) include means for determining an index which is representative of the local resistance of said segment during a systolic phase of a heartbeat, and said means for determining said second intermediate index includes means for determining an index (ID) which is representative of the distensibility of said artery during a systolic phase of a heartbeat.

In this case, equipment according to the invention preferably includes means for calculating said local stiffness index (Ira) according to the formula:

$$Ira = \frac{PAS - PAD}{PAM} \cdot \frac{RP \cdot ID}{RP + ID}$$

Said means for calculating said first intermediate index (RP) preferably include means for calculating according to the formula:

$$RP = \frac{PAM}{( I + J )}$$

$$with I = {\int }_{{t}_{1}}^{{t}_{2}} \vert \frac{d}{d t} ( \frac{1}{Δ Z} ) \vert d t and J = {\int }_{{t}_{2}}^{{t}_{3}} \vert \frac{d}{d t} ( \frac{1}{Δ Z} ) \vert d t ,$$

t₁ representing the appearance time of the base of the derivative of the impedance variation

$$( \frac{d}{d t} \frac{1}{Δ Z} )$$

from the start of said systolic phase,

t₂ representing the appearance time of the maximum of the derivative of the impedance variation

$$( \frac{d}{d t} \frac{1}{Δ Z} )$$

from the start of said systolic phase,

t₃ representing the appearance time of the intersection of the derivative of the impedance variation

$$( \frac{d}{d t} \frac{1}{Δ Z} )$$

and a straight line parallel to the x-axis passing through the point of the curve

$$( \frac{d}{d t} \frac{1}{Δ Z} )$$

at time t₁.

Said means for calculating said second intermediate index (ID) preferably include means for calculating according to the formula:

$$ID = PAM \frac{100 [ 2 ( J - I ) - ( J + I ) ]}{{( J + I )}^{2}}$$

Equipment according to the invention advantageously includes means for acquiring an electrocardiogram (ECG) signal from said patient, means for detecting each of the heartbeats (R) appearing on said electrocardiogram (ECG), means for activating said means for determining said indices subsequent to the detection of at least one heartbeat (R).

According to the invention, each index can be determined for one heartbeat. Alternatively, each of the indices can be determined for consecutive heartbeats, the value of the final index obtained corresponding to the average of the values of the consecutively determined indices. This enables the accuracy of the results obtained to be improved. According to yet another alternative, the impedance variation can be measured consecutively on consecutive heartbeats. A curve corresponding to the average of the impedance variation on the various heartbeats can then be obtained. The various indices can then be determined from this average curve. This embodiment likewise enables the accuracy of the results to be improved.

### 1. General Principle of the Invention

The general principle of the invention is based on the fact of determining two intermediate indices which are representative of a resistive characteristic and a capacitive characteristic, respectively, involved in the stiffening of an artery, and in then determining the local stiffness index of an artery wall based on the two predetermined intermediate indices.

The inventors discovered that the stiffening of an artery wall can in particular result from a so-called resistive characteristic, which is related to local or peripheral resistance of the artery, and a so-called capacitive characteristic, which is related to the elasticity of the artery. The knowledge of each of these resistive and capacitive characteristics of an artery enables a local stiffness index for said artery to be determined, which is particularly accurate and representative of reality.

Implementation of the invention therefore enables a global index to be obtained, on the one hand, which enables the level of local stiffness of an artery wall to be known, the recognition of which makes it possible to know if, along a segment, the artery is rather stiff or rather flexible, and two intermediate indices to be obtained, the recognition of which makes it possible to know the respective significance of a resistive characteristic and a capacitive characteristic of the artery involved in the local stiffness thereof.

Taking account of the results thus obtained makes it possible to have a more accurate picture of the stiffness of an artery and, in particular, to know the significance of the various characteristics which are at the source thereof, and to treat a patient more effectively, e.g., by administering thereto a treatment which is targeted at each of the characteristics involved in the stiffening of the arteries of same.

Furthermore, said two intermediate indices are obtained from the measurement, at a single point, of the electric impedance variation of a volume of blood flowing in a segment of the artery the stiffness of which one wishes to determine. Implementation of the invention is thus facilitated.

### 2. Example of a First Embodiment of Equipment for Implementing a Method According to the Invention

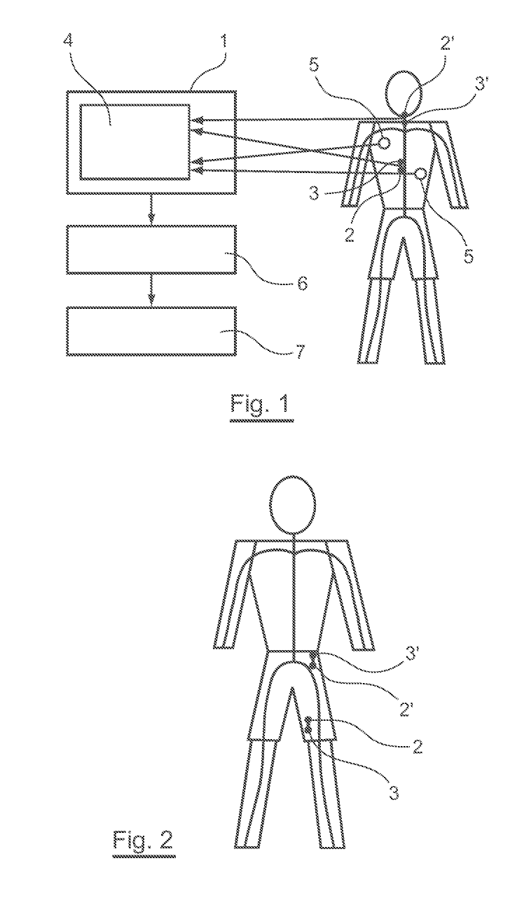

An embodiment of equipment for implementing a method according to the invention is introduced in connection with FIG. 1.

As shown in FIG. 1, such equipment includes two pairs of electrodes 2, 3 and 2′, 3′. Each of these pairs of electrodes includes an emitting electrode 2 or 2′ and a receiving electrode 3 or 3′. These pairs of electrodes are intended to be positioned on a patient such that they define a space inside of which an artery is located, the stiffness of which one wishes to determine, said electrodes defining an axis which is parallel to the major axis of said artery.

Such equipment likewise includes two other electrodes 5 intended to enable acquisition of the electrocardiogram signal of the patient.

Electrodes 2, 3, 2′, 3′ are connected to an inductometer 1 like those conventionally found on the market.

Such an inductometer includes synchronisation means 4 enabling an impedance signal measured in a volume (V) of blood flowing inside the portion (or segment) of the artery situated between the pairs of electrodes to be synchronised with the electrocardiogram signal.

This equipment further includes calculating means 6, e.g., a computer, which are connected to the inductometer 1 with a view to processing the signals output by same, and to calculate an index (Ira) representative of the local stiffness of the wall of the artery being studied, as will be described below.

It further includes means of displaying 7 the results obtained.

### 3. Example of a First Embodiment of a Method According to the Invention

A method for measuring the local stiffness index Ira of the wall of a conducting artery carrying the blood of a patient will now be described, in particular with reference to FIG. 4.

Such a method consists in positioning two pairs of emitting electrodes and receiving electrodes 2, 3 and 2′, 3′ on a patient such that same form an axis which is parallel to the axis of the artery the stiffness of which one wishes to determine and such that same define a space inside of which said artery is situated.

FIG. 1 indicates the location of the electrodes 2, 3, 2′, 3′ on the thorax, in order to study the aorta. Electrodes 2′ and 3′ are positioned at the base of the neck, on the same side, one above the other, without overlapping, and electrodes 2 and 3 are positioned below the sternum, one above the other, without overlapping.

FIG. 2 shows an exemplary location of these electrodes on the thigh in order to study the femoral artery. The positioning of these electrodes may of course be modified so as to cover other anatomical areas likely to contain a conducting artery the stiffness of which one wishes to study.

In addition, the present invention can be implemented in both humans and animals, provided that the signal acquired is representative of the blood flow in the conducting artery studied.

Electrocardiogram electrodes 5 are likewise put in place, e.g., on the thorax of the patient.

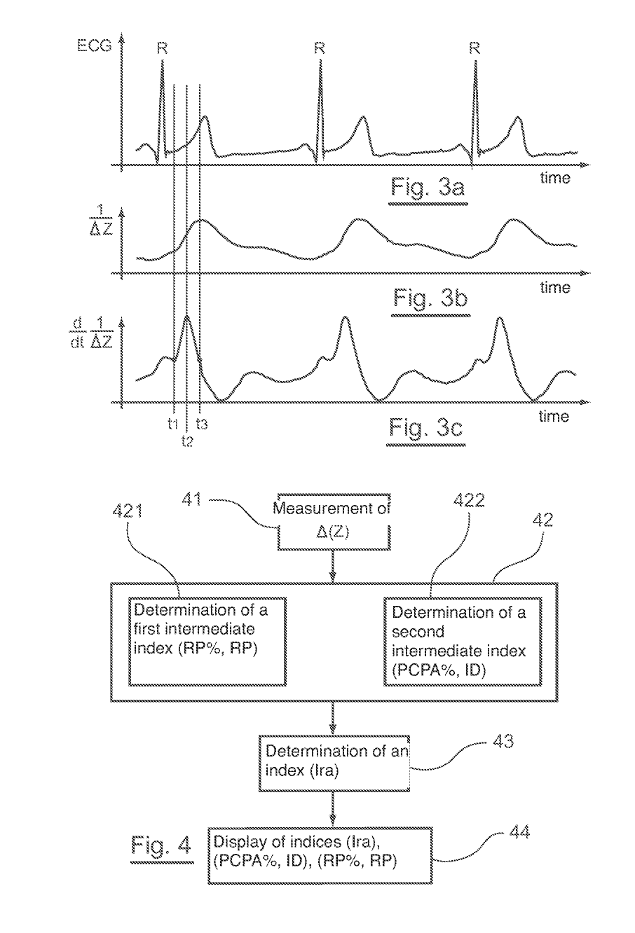

The electrodes 2, 3, 2′, 3′ and 5 are all connected to an inductometer 1 which enables:
an electric current of low intensity (of the order of 3 mA) and adjustable high frequency (of the order of 75 kHz) to be injected through the volume (V) of blood flowing in an artery segment positioned between the emitting electrodes 2, 2′ and the receiving electrodes 3, 3′;
measurement 41 of the impedance variation (ΔZ) in said volume of blood and acquisition of a signal representing the inverse of the impedance variation (ΔZ) (FIG. 3b);
acquisition of an electrocardiogram (ECG) signal of the patient (FIG. 3a).

It is noted that the use of the inductometer 1 is not accompanied by any unpleasant constraint for the patient (no compression, no limitation of movements) and does not have any use-related risk since the technique is non-invasive.

The signals output by the inductometer 1 are transmitted to calculating means, such as the computer 6, with a view to:
determining 42 a first intermediate index (RP %) and a second intermediate index (PCPA %) representative of a resistive characteristic and a capacitive characteristic, respectively, which are involved in the stiffening of the segment of the artery studied;
determining 43 an index (Ira) representative of the local stiffness of the wall of the artery, based on said first (RP %) and second (PCPA %) intermediate indices.

In this embodiment, based on the signals output by the inductometer 1, the computer 6 enables:
the first intermediate index (RP %) to be determined 421, which is representative of the peripheral resistance downstream from the arterial segment during a systolic phase of a heartbeat;
the second intermediate index (PCPA %) to be determined 422, which is representative of the capacity of the artery to store mechanical energy due to the deformation of the artery during the systolic phase of the heartbeat, and to restore same during the diastolic phase of the heartbeat.

Specifically, the computer 6 processes three signals with a view to determining the indices (Ira), (RP %) and (PCPA %):
the electrocardiogram (ECG) signal of the patient (FIG. 3a);
the signal representing the inverse of the impedance variation in the volume (V) of blood flowing in the portion of artery situated between the pairs of electrodes (FIG. 3b);
the signal representing the derivative of the inverse of the impedance variation (FIG. 3c).

The calculating means 6 determine the intermediate index (PCPA %) according to the formula:

$${PCPS}_{\%} = \frac{J - I}{J + I} \cdot 100$$

$$with$$

$$I = {\int }_{{t}_{1}}^{{t}_{2}} \vert \frac{d}{d t} ( \frac{1}{Δ Z} ) \vert d t$$

$$J = {\int }_{{t}_{2}}^{{t}_{3}} \vert \frac{d}{d t} ( \frac{1}{Δ Z} ) \vert d t$$

and with:

t₁ representing the appearance time of the base of the derivative of the impedance variation

$$( \frac{d}{d t} \frac{1}{Δ Z} )$$

from the start of said systolic phase,

t₂ representing the appearance time of the maximum of the derivative of the impedance variation

$$( \frac{d}{d t} \frac{1}{Δ Z} )$$

from the start of said systolic phase,

t₃ representing the appearance time of the intersection of the derivative of the impedance variation

$$( \frac{d}{d t} \frac{1}{Δ Z} )$$

and a straight line parallel to the x-axis passing through the point of the curve

$$( \frac{d}{d t} \frac{1}{Δ Z} )$$

at time t₁.

The calculating means 6 determine the intermediate index (RP %) according to the formula:

$${RP}_{\%} = \frac{K - I}{K} \cdot 100$$

K being a constant dependent on means implemented to measure the electric impedance variation ΔZ.

The constant K is obtained by carrying out a plurality of stiffness measurements of the wall of an artery in various patients:
by implementing a technique of the prior art taken as a reference, and
by implementing the technique according to the invention.

The value of constant K is then adjusted such that the technique according to the invention results in the obtainment of stiffness values which are equivalent to the values obtained according to the prior art. For example, said constant K may be equal to 5,000.

The calculating means 6 finally determine the index (Ira) based on the previously calculated intermediate indices (PCPA %) and (RP %), according to the formula:

Ira=(1−|PCPA %|)·RP %+(1−RP %)·|PCPA %|

The values of indices (Ira), (PCPA %) and (RP %) can then be displayed 44 on the display means 7, with a view to being analysable.

In an alternative, said display means 7 may be integrated into a virtual platform and the values may be transmitted remotely by the calculating means, or directly by the inductometer, so that the results can be analysed at a location remote from the one in which the measurements are carried out.

The parameters (Ira), (PCPA %) and (RP %) are calculated for a heartbeat. The inductometer 1 therefore includes synchronisation means 4 enabling synchronisation of the electrocardiogram (ECG) signal and the impedance signal measured in the volume (V) of blood flowing inside the portion of the artery situated between the pairs of electrodes.

The inductometer 1 or the calculating means 6 likewise include means which, by analysing the electrocardiogram signal, enable the occurrence of a heartbeat (R) to be detected and activation of the calculating means 6 to therefore be triggered, with a view to obtaining the value of the indices (Ira), (PCPA %) and (RP %).

In an alternative of this embodiment, the indices (Ira), (PCPA %) and (RP %) can correspond to the average of the curves produced for consecutive heartbeats (R). This can enable the accuracy of the results to be improved.

It was observed that the index (Ira) calculated for the aorta was strongly correlated with the velocity of propagation of the pulse wave measured by so-called reference tonometric techniques.

Implementation of the present invention enables not only the local stiffness index (Ira) of the wall of an artery segment to be provided but also two other intermediate indices (RP %) and (PCPA %) capable of assisting in diagnosis, for the purpose of specifying the characteristics responsible for stiffening of the arterial wall, and in a manner which is non-invasive, simple, fast and direct, without any operator handling procedure, and applicable to all of the conducting arteries.

The local stiffness index of the aortic wall is provided by the index (Ira), and the relative significance of the resistive component and the capacitive component responsible for the arterial stiffness can be quantified and assessed by a person skilled in the art, by interpreting the intermediate indices (RP %) and (PCPA %).

It was thus observed experimentally that the values of the intermediate indices (RP %) and (PCPA %) may be indexed into various classes illustrating the state of the arteries, and mentioned herein for purely illustrative purposes:
(RP %) lower than 50% and (PCPA %) higher than 5% indicate low peripheral resistance and high aortic elasticity;
(RP %) lower than 50% and (PCPA %) lower than −5% indicate low peripheral resistance and low aortic elasticity;
(RP %) higher than 50% and (PCPA %) lower than −5% indicate high peripheral resistance and low aortic elasticity;
(RP %) higher than 50% and (PCPA %) higher than 5% indicate high peripheral resistance and high aortic elasticity.

Taking account of the results thus obtained makes it possible to have a more accurate picture of the overall stiffness of a segment of an artery and to know the significance of the various characteristics which are at the source thereof. A patient can then be treated more effectively by administering thereto a treatment which is targeted at each of the characteristics involved in the stiffening of the arteries of same.

Implementation of the technique according to the invention enables the stiffness of any conducting artery to be determined at any given moment. It can likewise make it possible to follow the development thereof by consecutive measurements. It can likewise make it possible to evaluate the effect, e.g., of administering a drug treatment, performing internal or external physical treatment or any other operation on the stiffness of a patient's artery.

The invention can likewise be implemented as a “screening” type test for developing new molecules likely to become drugs.

It can be implemented in both humans and animals.

### 4. Example of a Second Embodiment of Equipment for Implementing a Method According to the Invention

Equipment according to a second embodiment is differentiated from that of the first embodiment in that it further includes means of measuring the arterial pressure in the systolic phase PAS, means of measuring the arterial pressure in the diastolic phase PAD and means of calculating the average arterial pressure PAM.

The means of determining the indices, which include the computer 6, are programmed to execute different calculation formulas, as will be explained in greater detail hereinbelow.

### 5. Example of a Second Embodiment of a Method According to the Invention

This second embodiment differs from the first embodiment in that it includes a step of measuring the arterial pressure in the systolic phase PAS, a step of measuring the arterial pressure in the diastolic phase PAD and a step of calculating the average arterial pressure PAM.

It likewise differs in that, based on the signals output by the inductometer 1, the computer 6 enables:
a first intermediate index (RP) to be determined 421, which is representative of the local resistance of the arterial segment during a systolic phase of a heartbeat;
a second intermediate index (ID) to be determined 422, which is representative of the distensibility of the artery.

Specifically, the computer 6 process three signals with a view to determining the indices (Ira), (RP) and (ID):
the electrocardiogram (ECG) signal of the patient (FIG. 3a);
the signal representing the inverse of the impedance variation in the volume (V) of blood flowing in the portion of artery situated between the pairs of electrodes (FIG. 3b);
the signal representing the derivative of the inverse of the impedance variation (FIG. 3c).

The impedance variation or the derivative of the inverse of the impedance variation of the blood flowing in the segment of an artery can be compared to the measurement of the kinetic energy flowing inside the conducting artery studied.

During the start of the systolic phase, the blood flowing in the segment has a first kinetic energy EC 1 and the artery stores mechanical energy EM1. During the end of the systolic phase, the blood flowing in the segment has a second kinetic energy EC2 and the artery stores mechanical energy EM2. During the diastolic phase, the artery restores a total mechanical energy equal to the sum of the mechanical energies EM1 and EM2.

The first intermediate index (RP) is written:

$$RP = \frac{PAM}{( EC 1 + EC 2 )}$$

The first kinetic energy EC1 is equal to I. The second kinetic energy EC2 is equal to J. Therefore, it is deduced therefrom that:

$$RP = \frac{PAM}{( I + J )}$$

The calculating means 6 determine the first intermediate index (RP), according to the formula:

$$RP = \frac{PAM}{( I + J )}$$

$$with$$

$$I = {\int }_{{t}_{1}}^{{t}_{2}} \vert \frac{d}{d t} ( \frac{1}{Δ Z} ) \vert d t and J = {\int }_{{t}_{2}}^{{t}_{3}} \vert \frac{d}{d t} ( \frac{1}{Δ Z} ) \vert d t ,$$

t₁ representing the appearance time of the base of the derivative of the impedance variation

$$( \frac{d}{d t} \frac{1}{Δ Z} )$$

from the start of said systolic phase,

t₂ representing the appearance time of the maximum of the derivative of the impedance variation

$$( \frac{d}{d t} \frac{1}{Δ Z} )$$

from the start of said systolic phase,

t₃ representing the appearance time of the intersection of the derivative of the impedance variation

$$( \frac{d}{d t} \frac{1}{Δ Z} )$$

and a straight line parallel to the x-axis passing through the point of the curve

$$( \frac{d}{d t} \frac{1}{Δ Z} )$$

at time t₁.

The second intermediate index (ID) is written:

$$ID = \frac{PAM}{( EM 1 + EM 2 )} = PAM \frac{100 [ 2 ( J - I ) - ( J + I ) ]}{{( J + I )}^{2}}$$

The calculating means 6 determine the second intermediate index (ID) according to the formula:

$$ID = PAM \frac{100 [ 2 ( J - I ) - ( J + I ) ]}{{( J + I )}^{2}}$$

The calculating means 6 finally determine the index (Ira) based on the first (RP) and second (ID) previously calculated intermediate indices, according to the formula:

$$Ira = \frac{PAS - PAD}{PAM} \cdot \frac{RP \cdot ID}{RP + ID}$$

The local stiffness index of the aortic wall is provided by the index (Ira), and the relative significance of the resistive component and the capacitive component responsible for the arterial stiffness may be quantified and assessed by a person skilled in the art, by interpreting the intermediate indices (RP) and (PCPA).

It was thus observed experimentally that the values of the intermediate indices (RP) and (ID) may be indexed into various classes illustrating the state of the arteries, and mentioned herein for purely illustrative purposes:
(RP) lower than 5 and (ID) higher than 20 indicate low local resistance and high aortic distensibility;
(RP) lower than 5 and (ID) lower than 5 indicate low local resistance and low aortic distensibility;
(RP) higher than 20 and (ID) lower than 5 indicate high local resistance and low aortic distensibility;
(RP) higher than 20 and (ID) higher than 20 indicate high local resistance and high aortic distensibility.

1. A method for measuring the local stiffness index (Ira) of the wall of a conducting artery carrying the blood of a patient, wherein said method includes at least:
a step of measuring, at a single measurement point, the electric impedance variation (ΔZ) of a volume (V) of the blood flowing in a segment of said artery;
a step of determining a first intermediate index (RP %, RP) representative of a resistive characteristic involved in the stiffening of said wall, and a second intermediate index (PCPA %, ID) representative of a capacitive characteristic involved in the stiffening of said wall, the first (RP %, RP) and second (PCPA %, ID) intermediate indices being obtained from the measure of the electric impedance variation (ΔZ); and
a step of determining said local stiffness index (Ira) based on said first (RP %, RP) and second (PCPA %, ID) intermediate indices.

2. The method according to claim 1, wherein said first (RP %) is an index representative of the peripheral resistance downstream from said segment during a systolic phase of a heartbeat, and said second intermediate index (PCPA %) is an index representative of the capacity of said artery to store mechanical energy due to the deformation of said artery during said systolic phase of said heartbeat, and to restore same during the diastolic phase of said heartbeat.

3. The method according to claim 2, wherein said step of determining said local stiffness index (Ira) includes a step of calculating according to the formula:
Ira=(1−|PCPA %|)·RP %+(1−RP %)·|PCPA %|

4. The method according to claim 3, wherein it includes a step of calculating said second intermediate index (PCPA %) according to the formula:
$${PCPA}_{\%} = \frac{J - I}{J + I} \cdot 100$$
$$with I = {\int }_{{t}_{1}}^{{t}_{2}} \vert \frac{d}{d t} ( \frac{1}{Δ Z} ) \vert d t and I = {\int }_{{t}_{2}}^{{t}_{3}} \vert \frac{d}{d t} ( \frac{1}{Δ Z} ) \vert d t ,$$
t₁ representing the appearance time of the base of the derivative of the impedance variation
$$( \frac{d}{d t} \frac{1}{Δ Z} )$$
from the start of said systolic phase,
t₂ representing the appearance time of the maximum of the derivative of the impedance variation
$$( \frac{d}{d t} \frac{1}{Δ Z} )$$
from the start of said systolic phase, and
t₃ representing the appearance time of the intersection of the derivative of the impedance variation
$$( \frac{d}{d t} \frac{1}{Δ Z} )$$
and a straight line parallel to the x-axis passing through the point of the curve
$$( \frac{d}{d t} \frac{1}{Δ Z} )$$
at time t₁.

5. The method according to claim 4, wherein it includes a step of calculating said first intermediate index (RP %) according to the formula:
$${RP}_{\%} = \frac{K - I}{K} \cdot 100$$
K being a constant dependent on means implemented to carry out said step of measuring the electric impedance variation (ΔZ).

6. The method according to claim 1, wherein said first intermediate index is an index (RP) which is representative of the local resistance of said segment during a systolic phase of a heartbeat, and said second intermediate index is an index (ID) which is representative of the distensibility of said artery during a systolic phase of a heartbeat.

7. The method according to claim 6, wherein it includes a step of measuring the arterial pressure in the systolic phase (PAS), the arterial pressure in the diastolic phase (PAD), and calculating the average arterial pressure (PAM).

8. The method according to claim 7, wherein said step of determining said local stiffening index (Ira) includes a step of calculating according to the formula:
$$Ira = \frac{PAS - PAD}{PAM} \cdot \frac{RP \cdot ID}{RP + ID}$$

9. The method according to claim 8, wherein it includes a step of calculating said first intermediate index (RP) according to the formula.
$$RP = \frac{PAM}{( I + J )}$$
$$with$$
$$I = {\int }_{{t}_{1}}^{{t}_{2}} \vert \frac{d}{d t} ( \frac{1}{Δ Z} ) \vert d t and J = {\int }_{{t}_{2}}^{{t}_{3}} \vert \frac{d}{d t} ( \frac{1}{Δ Z} ) \vert d t ,$$
t₁ representing the appearance time of the base of the derivative of the impedance variation
$$( \frac{d}{d t} \frac{1}{Δ Z} )$$
from the start of said systolic phase,
t₂ representing the appearance time of the maximum of the derivative of the impedance variation
$$( \frac{d}{d t} \frac{1}{Δ Z} )$$
from the start of said systolic phase, and
t₃ representing the appearance time of the intersection of the derivative of the impedance variation
$$( \frac{d}{d t} \frac{1}{Δ Z} )$$
and a straight line parallel to the x-axis passing through the point of the curve
$$( \frac{d }{d t} \frac{1}{Δ Z} )$$
at time t₁.

10. The method according to claim 1 wherein it includes as step of calculating said second intermediate index (ID) according to the formula:
$$ID = PAM \frac{100 [ 2 ( J - I ) - ( J + I ) ]}{{( J + I )}^{2}}$$

11. The method according to claim 1, wherein it includes a step of acquiring an electrocardiogram (ECG) signal from said patient, and a step of synchronising said electrocardiogram (ECG) signal and said impedance variation (ΔZ).

12. The method according to claim 1, wherein it includes a plurality of:
steps of determining said first (RP %, RP) and said second (PCPA %, ID) intermediate indices;
steps of determining said local stiffness index (Ira) based on said first (RP %, RP) and said second (PCPA %, ID) intermediate indices, and
said determination steps being carried out during consecutive heartbeats (R), said method also including a step of calculating the average of each of said indices (Ira, RP %, RP, PCPA %, ID) during said heartbeats (R).

13. The method according to claim 1, wherein it includes a plurality of steps of measuring, at a single measurement point, the electric impedance variation (ΔZ) of a volume (V) of blood flowing in a segment of said artery, each of said measurements being carried out on different heartbeats (R), said method also including a step of determining the average impedance variation on said heartbeats and a step of determining said first (RP %, RP) and said second (PCPA %, ID) intermediate indices based on said average.

14. The method according to claim 1, wherein it includes a step of displaying said local stiffness index (Ira) of the wall of a conducting artery, and a step of displaying said first (RP %, RP) and said second (PCPA %, ID) intermediate indices.

15. Equipment for implementing the method for determining the local stiffness index (Ira) of the wall of a blood-carrying conducting artery of a patient according to claim 1, wherein it includes:
means for measuring, at a single measurement point, the electric impedance variation (ΔZ) of a volume (V) of the blood flowing in a segment of said artery;
means for determining a first intermediate index (RP %, RP) representative of a resistive characteristic involved in the stiffening of said wall, and a second intermediate index (PCPA %, ID) representative of a capacitive characteristic involved in the stiffening of said wall; and
means for determining said local stiffness index (Ira) based on said first (RP %, RP) and second (PCPA %, ID) intermediate indices.

16. The equipment according to claim 15, wherein said means for determining said first intermediate index (RP %) include means for determining an index representative of the peripheral resistance downstream from said segment during a systolic phase of a heartbeat, and said means for determining said second intermediate index (PCPA %) include means for determining an index representative of the capacity of said artery to store mechanical energy due to the deformation of said artery during said systolic phase of said heartbeat and to restore same during the diastolic phase of said heartbeat.

17. The equipment according to claim 16, wherein it includes means for calculating said local stiffness index (Ira) according to the following formula:
Ira=(1−|PCPA %|)·RP %+(1−RP %)·|PCPA %|

18. The equipment according to claim 17, wherein said means for calculating said second intermediate index (PCPA %) include means for calculating according to the formula:
$${PCPA}_{\%} = \frac{J - I}{J + I} \cdot 100$$
$$with$$
$$I = {\int }_{{t}_{1}}^{{t}_{2}} \vert \frac{d }{d t} ( \frac{1}{Δ Z} ) \vert d t and J = {\int }_{{t}_{2}}^{{t}_{3}} \vert \frac{d }{d t} ( \frac{1}{Δ Z} ) \vert d t ,$$
t₁ representing the appearance time of the base of the derivative of the impedance variation
$$( \frac{d }{d t} \frac{1}{Δ Z} )$$
from the start of said systolic phase,
t₂ representing the appearance time of the maximum of the derivative of the impedance variation
$$( \frac{d }{d t} \frac{1}{Δ Z} )$$
from the start of said systolic phase, and
t₃ representing the appearance time of the intersection of the derivative of the impedance variation
$$( \frac{d }{d t} \frac{1}{Δ Z} )$$
and a straight line parallel to the x-axis passing through the point of the curve
$$( \frac{d }{d t} \frac{1}{Δ Z} )$$
at time t₁.

19. The equipment according to claim 18, wherein said means for calculating said first intermediate index (RP %) include means for calculating according to the formula:
$${RP}_{\%} = \frac{K - I}{K} \cdot 100$$
K being a constant dependent on means implemented to carry out said step of measuring the electric impedance variation (ΔZ).

20. The equipment according to claim 15, wherein said means for determining said first intermediate index (RP) include means for determining an index which is representative of the local resistance of said segment during a systolic phase of a heartbeat, and said means for determining said second intermediate index includes means for determining an index (ID) which is representative of the distensibility of said artery during a systolic phase of a heartbeat.

21. The equipment according to claim 20, wherein it includes means for calculating said local stiffness index (Ira) according to the formula:
$$Ira = \frac{PAS - PAD}{PAM} \cdot \frac{RP \cdot ID}{RP + ID}$$

22. The equipment according to claim 21, wherein said means for calculating said first intermediate index (RP) include means for calculating according to the formula:
$$RP = \frac{PAM}{( I + J )}$$
$$with$$
$$I = {\int }_{{t}_{1}}^{{t}_{2}} \vert \frac{d }{d t} ( \frac{1}{Δ Z} ) \vert d t and J = {\int }_{{t}_{2}}^{{t}_{3}} \vert \frac{d }{d t} ( \frac{1}{Δ Z} ) \vert d t ,$$
t₁ representing the appearance time of the base of the derivative of the impedance variation
$$( \frac{d }{d t} \frac{1}{Δ Z} )$$
from the start of said systolic phase,
t₂ representing the appearance time of the maximum of the derivative of the impedance variation
$$( \frac{d }{d t} \frac{1}{Δ Z} )$$
from the start of said systolic phase, and
t₃ representing the appearance time of the intersection of the derivative of the impedance variation
$$( \frac{d }{d t} \frac{1}{Δ Z} )$$
and a straight line parallel to the x-axis passing through the point of the curve
$$( \frac{d }{d t} \frac{1}{Δ Z} )$$
at time t₁.

23. The equipment according to claim 22, wherein said means for calculating said second intermediate index (ID) includes means of calculating according to the formula:
$$ID = PAM \frac{100 [ 2 ( J - I ) - ( J + I ) ]}{{( J + I )}^{2}}$$

24. The equipment according to claim 15, wherein it includes means for acquiring an electrocardiogram (ECG) signal from said patient, means for detecting each of the heartbeats R appearing on said electrocardiogram (ECG), means for activating said means for determining said indices subsequent to the detection of at least one heartbeat (R).
